(19) United States
(12) Patent Application Publication
(10) Pub. No.: US 2026/0266924 A1
(43) Pub. Date: Sep. 10, 2026

(54) APPARATUS AND METHODS FOR IMAGE-GUIDED ROBOTIC SURGICAL INTERVENTIONS

(71) Applicant: Worcester Polytechnic Institute, Worcester, MA (US)

(72) Inventors: Gregory Fischer, Needham, MA (US); Christopher Nycz, Holden, MA (US); Katie Gandomi, Weston, FL (US); Paulo Carvalho, Piata (BR); Zhanyue Zhou, Brighton, MA (US)

(21) Appl. No.: 19/491,313

(22) PCT Filed: Jun. 7, 2024
(86) PCT No.: PCT/US2024/032919

§ 371 Date: Dec. 8, 2025

Related U.S. Application Data
(60) Provisional application No. 63/472,106, filed on Jun. 9, 2023.
(60) Provisional application No. 63/472,108, filed on Jun. 9, 2023.
(60) Provisional application No. 63/472,109, filed on Jun. 9, 2023.

(51) Int. Cl. G01R 33/00; G01R 33/421; G01R 33/54; H02N 2/00; H02N 2/14; H10N 30/20; H10N 30/50

(52) U.S. Cl. CPC G01R 33/0041; G01R 33/007; G01R 33/421; G01R 33/546; H02N 2/0095; H02N 2/14; H10N 30/2023; H10N 30/50

(57) ABSTRACT

The present invention comprises unique piezo elements which can be configured an controlled to provide motion in any direction. Included are rotor motors capable of being combined around one or more concentric tubes or shafts wherein the motors allow control over each individual tube or shaft. These motors may be used in the manufacture of end effectors for robots. Embodiments include an active surgical drape incorporating means for attaching end effectors and passing through electrical signals, fluids, and mechanical parts. All of the foregoing can function inside an actively scanning MRI.

### PRIORITY CLAIM

This application claims priority to U.S. provisional patent application No. 63/472,106 filed on Jun. 9, 2023, and U.S. provisional patent application No. 63/472,108 filed on Jun. 9, 2023, and U.S. provisional patent application No. 63/472,109 filed on Jun. 9, 2023, the contents of which are expressly incorporated by reference.

### CROSS-REFERENCES AND INCORPORATION BY REFERENCE

This application cross references and incorporates by reference the content of Published United States patent applications number US 2011/0077504 A1; US 2012/0265051; US 2014/0107659 A1; US 2019/0223972 A1; US 2019/0054275 A1; US 2018/0049826 A1; US 2017/0325906 A1; and U.S. Ser. No. 14/056,205 The contents of all references cited herein are expressly incorporate by reference.

### GOVERNMENT SUPPORT

This invention was made with government support under grant R01CA166379 awarded by the National Institutes of Health, the government has certain rights in the invention.

### BACKGROUND

Technologies for piezoelectric actuation are described and, more particularly, such technologies which can be used for creating and controlling motion of robotic devices intended to operate in or near an MRI scanner for medical application. The technologies may be used for a variety of other applications, including those with sensitivity to electromagnetic fields or electrical noise, aerospace and explosive environments.

### SUMMARY

An omnidirectional piezo motor is provided and comprises at least two piezo electric stacks, each stack comprising a plurality of piezo electric crystals, each crystal having a conductive and a non-conductive region, wherein the stacks are oriented so the non-conductive regions of each stack are adjacent to each other. A controller electrically connected to each of the at least 3 stacks provides a controlling electrical signal to each piezo electric stack. In one aspect, the stacks are controlled via waveforms, and the waveforms may be sine or cosine.

In another aspect, the multiple piezo electric stacks are combined and controlled to allow piezo electric static to enact a desired motion in more than a single axis. The piezo electric motor may be comprised of 4 pillars and the pillars capped with a purpose specific tip. In a further aspect, the pillars can be coupled and actuated in unique ways to enact a driving force in an arbitrary direction within a plane tangent to a point on the actuator tip. In this aspect, the pillars can be coupled and energized in unique ways to enact a driving force within any contact surface in contact with the actuator tip and the force is acting in a direction tangent to that point of contact and perpendicular to the normal axis of contact. Still further, the contact surface being acted upon is a known geometric shape, including but not limited to circles, spheres, cylinders, rectangular prisms and others.

In yet another aspect, the piezoelectric crystals are arranged radially as a stator and act upon a rotor. The center of the motor is hollow, and multiple radial actuators can be arranged and controlled in concert to generate complex motions. Multiple motors may be arranged on a single axis to function in a differential motion system. The hollow center of the motor may be designed to hold a tool or instrument.

In another embodiment, of the omnidirectional piezo motor, a first piezo motor acts on a first drive shaft which is coupled to a first control element, and a second piezo motor acts on a second drive shaft which is coupled to a tube concentric with the first control element, wherein the tube and the control element are capable of individually controlled motion. This may further comprise one or more additional motors each acting on an additional driven tube, all of the tubes being concentric with the first control element and the concentric tube.

In a method of operating the omni-direction motor, the different piezo crystals, stacks, and arrangements are energized in concert in a coordinated fashion such to facilitate a coordinated, complex motion.

A system of tools and or improvements to enable MRI guided interventions are also provided. The system comprises a modular and configurable architecture separating functional elements for MRI guided technologies. This includes a modular controller configuration, such that swappable card based drivers can be interchanged to operate a variety of MRI compatible actuators, sensors, and interface devices, an MRI compatible central computing/controller system, and an MRI compatible communication route through the patch panel of the control room to connect to a non-MRI compatible user interface. The user interface could be a standalone computer or directly tied to the control computer.

A module for the system may act as an MRI compatible motor driver, specifically paying attention to driving signal and communication signal generation such as not to emit intolerable interference into the scanner. In one aspect the module is for controlling a piezo electric ceramic motor or an arrangement of piezoelectric motors. The module can operate and interface with an imaging coil within the scanner or a robot within the scanner, wherein the imaging coil is mounted on the robot to move with the robots range of motion specifically to effect the image optimally based on the motion of the robot. In one aspect, the module registers the position of equipment within the imaging space. The module may be a contrast agent filled fiducial such that it can be segmented in the imaging space. In one aspect, the module is permanently mounted with a known geometric relationship to the robots coordinate system such that the robots coordinate system can be registered within an imaging volume. The robot system or base system can be attached to at least one other structure in the scanner room such as the bed rail, such that the fiducial can be used to track the relative position of said structure to the imaging space.

In another aspect, the module is a user interface or display wherein the module could be an alternative to a computer screen. In one embodiment, the module is a virtual reality headset, that allows the user to view representative imaging in 3-dimensional space, and utilize head tracking to optimize availability of data and minimize required use of hands. In another embodiment, the module is an augmented reality headset such that the user can view additional information from the system, as well as transparently see the environment around or in front of them. In still another embodiment, the module is a sensing receiver to provide the control system information from various devices, wherein the sensor may provide information on thermal properties of an ongoing procedure, or the sensor is a thermistor, thermal probe, or MRI based temperature readings.

A method is provided for operating the system in which the system and modules are controlled to perform complex operations by utilizing some or all of the subsystem components and modules in a coordinated fashion. Thermal information is utilized to perform closed loop thermal control of a thermal tool such as an ablation probe.

In another embodiment, a module's physical properties can be influenced by a scanner magnetic field and alteration thereof. The module may respond to motion or vibration induced upon it by integrated control of the scanner system magnetic field, such as controlling frequency, intensity or direction of magnetic field.

The module may generate heat in response to the MRI field modulation.

In another embodiment, a modular end effector is added to the robot as end of arm tooling. The module may be a swappable end effector capable of being replaced with a minimum of tools and the module may be swappable during a procedure to expand the capabilities of the robotic system. In one aspect, the module is a biopsy needle, bone drill, or other device that either is, or replicates the function of standard surgical tools. In another aspect, the module is a coaxial shielding extensible across the coupling of multiple physical modules to maintain an RF shielding barrier. In still another aspect, the module is a conductive flexible sleeve and a mating agent. The module may contain components or structure beneficial to be MRI shield, which can interface and extend the MRI shield through application of the interface. The module can be a needle driver, wherein the needle driver comprises hollow core piezo electric motors with the shaft advancement mechanism contained within the hollow core drive.

In still another embodiment, the module comprises a sterile drape incorporating one or more active or inactive elements and features to allow functionality to penetrate the sterile barrier, wherein the drape comprises an interface and connection point, having at least one conductor, actuator, sensor or pass through. This module may be disposable or single use, and designed to be discarded or reprocessed after each patient. In one aspect, the module is customized for specific patients or procedures. The interface for the active surgical drape can be used as a common interface for swappable end effectors.

### DESCRIPTION OF THE FIGURES

FIG. 1A is a drawing of piezo crystal

FIG. 1B is drawing showing a top and bottom of 4 piezo crystals-

FIG. 1C is a drawings of the piezo crystal of FIG. 1B showing the electrical inputs.

FIG. 1D is a drawing showing 4 stacks of piezo crystals.

FIG. 1E is a drawing of a piezo stack with a rotor.

FIG. 1F is a drawing showing prior art piezo motion.

FIG. 1G is a drawing showing an array of piezo stacks with a rotor.

FIG. 1H is a drawing of cylindrical piezo motor.

FIG. 1I is a drawing of a spherical piezo motor.

FIG. 2A is a drawing of a radial piezo motor.

FIGS. 2B-E are drawings of a radial piezo motor.

FIG. 2F is a cutaway drawing of a radial piezo motor.

FIGS. 2 G-J are drawings of a radial piezo motor.

FIGS. 2K-M are drawings of tools attached to a radial piezo motor.

FIGS. 2N-Q are drawings showing a tool under control of two radial piezo motors.

FIG. 3A is a drawing of the components of a surgical robotic system.

FIG. 3B is a drawing showing orientation of components with respect to a patient.

FIG. 4 is a drawing showing fiducial placement.

FIG. 5 is a drawing showing swappable effectors.

FIG. 6 is a drawing of a coaxial probe.

FIG. 7A is a drawing of hollow core motors driving a shaft

FIG. 7B is a drawing of a plurality of hollow core motors driving concentric shafts and tubes.

FIG. 8 is a drawing of an active surgical drape.

FIGS. 9-A-C are photos of a control cable.

FIG. 10A is a rendering of a control box

FIG. 10B is a photo of a control box

FIG. 11 is a drawing showing the control architecture

FIG. 12 is a rendering of a patient inside and MRI with the present invention

FIG. 13 is a drawing showing a movable imaging coil.

### DETAILED DESCRIPTION OF THE INVENTION

This application is focused on technologies for piezoelectric actuation. In one exemplary application, these technologies can be used for creating and controlling motion of robotic devices intended to operate in or near an MRI scanner for medical application. They also may be used for a variety of applications, including those with sensitivity to electromagnetic fields or electrical noise. Further example applications aerospace and explosive environments.

The noted embodiments are only exemplary embodiments and do not preclude other configurations or approaches. Where the text explicitly calls out piezoelectric actuation principles which include piezoelectric ceramic materials such as, but not limited to, PZT, this approached outlined in the invention is not limited to only piezoelectric actuation. The approach can also, for example, be applied to electroactive polymers or optically excited actuators such as but not limited to, PZLT or similar. It can also apply to pneumatic, hydraulic, or other method of energy transfer. The motion of the motion inducing elements may include harmonic or non-harmonic motion, and may include standing wave or traveling wave motion.

### Example 1: Omni-Directional Legged Piezo Motor

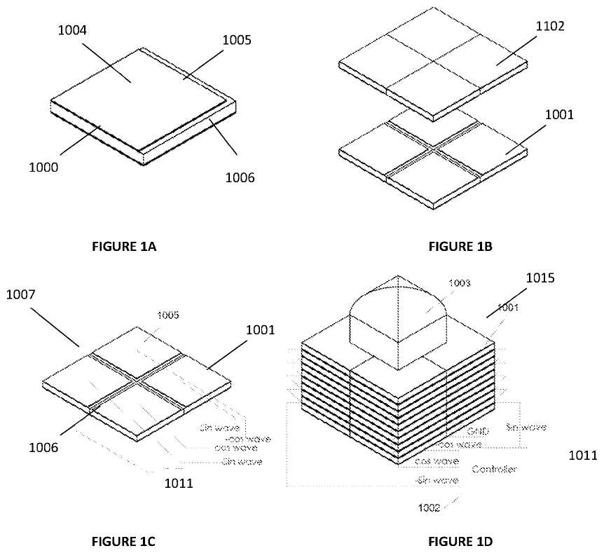

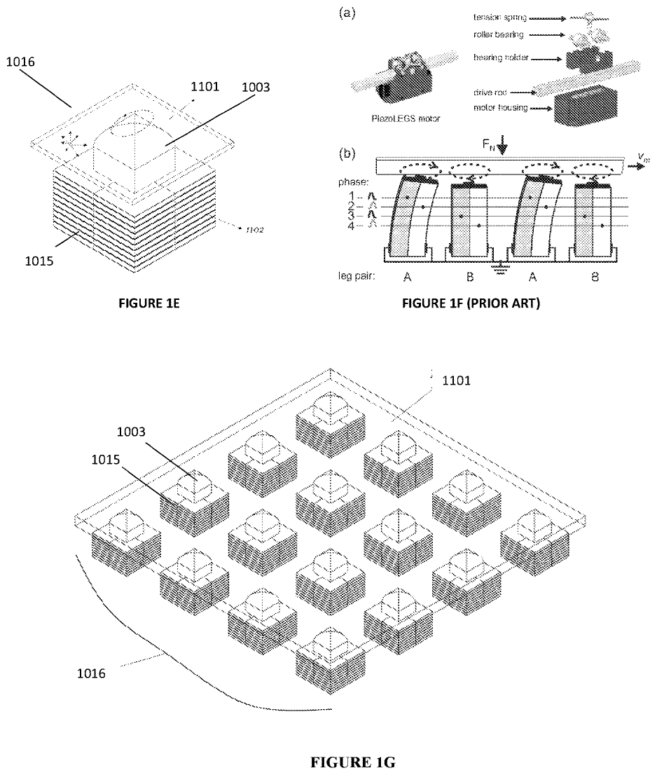

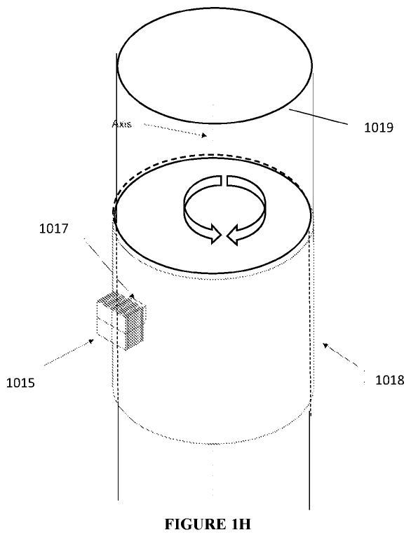

We describe a mechanism capable of motion in one or more directions which may include bending, shrinking, or elongating. Referring to FIGS. 1A, an embodiment of said mechanism includes a piezoelectric stack (1001, 1102) (or other material of controllable strain such as a dielectric elastomer, electroactive polymer, photostrictive material, or other) forming, at least, a three member group (four in the embodiment shown) which are mechanically coupled. In one embodiment the stack is a piezoelectric stack comprised of a plurality of piezo electric crystals (1000) stacked on top of each other to form piezoelectric stack (1102). The crystals having a positive electrical input (1010) and a ground (1011), the positive electrical input (1010) being driven by a controller 1002 that induces and expansion or contraction in the stack depending on the wave form of the current to each piezo electric ceramic unit (1000). Piezo electric ceramic unit can be any piezo electric ceramic but are commonly lead zirconate titanate (PZT), barium titanate (BT), and strontium titanate (ST) are the most widely used piezoelectric ceramic materials. The at least two stacks are connected to controller (1002) and may be activated by said controller individually or in combination.

Excitation of the appropriate stacks in sequence, together or in another periodic or a cyclic manner such as but not limited to sine or cosine waves by the controller (1002) which can be electric, optical, pneumatic, hydraulic or via some other method of energy transference, leads to repeatable motion of the stack. The stack may be terminated or otherwise include a component used to mediate, or otherwise participate, in the mechanical coupling, or transference of energy, between said stack (1102) and another component hereby referred to as rotor (1101) which can be of any geometry but is most commonly a sphere, rectangular prism, cylinder, rod, plate, or other shape. The contact, or transference of energy, between the stack (1001, 1102) and rotor (1101) leads to motion of one, or both, components. Elements of the stack may be plated, or otherwise contain one or more layers of material (1005), used for energetic coupling between the controller and said component of the stack. This invention does not exclude the possibility of combining two, or more, said stacks into a single device (1201). Said stacks may or may not be in the same plane of actuation or connected, in any way, with the same rotor (1101, 1202).

Referring to FIGS. 1a, 1B and 1C which show an example of a square piezo electric ceramic unit 1000 with a conductor (1005) (e.g. copper) on the top and bottom surfaces. In one embodiment, one side (e.g. the ground side 1011) is fully covered with the copper conductor 1005, while the other side (e.g. the excited side 1001) is partially covered (1004) (e.g. 90% covered) this leaving 2 edges (1006) etched on the partially covered side.

Referring to FIG. 1C which shows a 4 part piezo electric ceramic unit 1007 comprising 4 square piezo electric ceramic units 1000 together, wherein the excited surface will be input 4 phase changed signals (sine wave, −sin wave, cos wave, −cos wave) and without any shortcut or signal disturbance. And the return or grounds (GND) 1011 are all electrically connected together.

FIG. 1D is an exemplary stack (1015) showing the 4-part piezo electric ceramic units 1007 stacked on top of each other, with the excited sides 1006 facing together. In this example 10 such layers of 4-part units (1007) are stacked. A tip (1003) is placed upon the layers as an interface to the component that moves relative to the element. For multi-degrees of freedom (DOF) motion this may be a contact point, wherein in 1-DOF motion the contact may be a line or a point.

FIG. 1E shows an exemplary stack 1016 configured to move a planar surface in two degrees of freedom. For example, by controlling the signals there will be a relative motion of the surface with respect to the stack in any direction in X-Y plane. A preload would be applied to compress the rotor surface (1101) into the contacting tip (1003) of stack (1102) or vice versa. The contacting tip (1003) of stack 1102 may be a point or rounded surface, and the surface finish of the tip and the contacting surface of the rotor may be optimized for enhancing the motion or force produced.

FIG. 1F shows a commercially available bi-morph piezoelectric actuator with 4 legs that cause linear motion. An alternative embodiment of the stack described in FIG. 1F is to put a second set of bimorph actuators on top of a first set, and rotated with respect to the first such that one set causes motion in one direction and the other set causes motion in a directional different that the first direction. In one configuration, the amount of rotation of the second set is 90 degrees and the second direction of motion is orthogonal to the first. It will be appreciated that the first and second configuration can be any angle as long as they are not the same direction. By independently controlling both sets of electrodes and having a point (rather than line) based tip in contact with the moving surface, 2-DOF motion may be achieved. Alternatively, bi-morph actuators may be configured in at least 2 different orientations (e.g. half of the elements rotated 90 degrees from the others) such that they produce motion in different directions when actuated alone, and by coordinating motion the rotor surface may be moved in 2 or more directions. For all described configurations alternate embodiments utilizing different stack structures are not excluded from this invention.

Referring to FIG. 1G, the stacks 1016 may be arranged such that there are one or more such components in contact with the rotor surface 1101 surface that move relative to them. At least 4 such elements in a row or column can make sure the system can output stable power, with the average driving force in almost a straight line (similar to linear 4 cylinder engines in cars). A preload can be applied to 1015 and/or 1101 to apply a contact force between the stacks 1015 and the rotor surface 1101. The elements may be organized in rows or columns, or in a more arbitrary pattern. The stacks may be coordinated to enable 2-DOF motion in the plane of the surface 1101, and also may be configured and controlled as to enable 3-DOF motion in that plane which also includes rotation about an axis normal to the surface.

FIG. 1H shows an example of a cylindrical configuration 1020, wherein in this drawn example it is driven by one piezo element 1016. The piezo element 1016 can be unidirectional or bi-directional. One or more such piezo elements 1016 may be in contact with the friction surface of the cylinder 1017 at the respective contact point 1018 of each piezo element 1016. A preload may apply sufficient load to the contact point 1018 to enable frictional driving force. The cylinder 1017 may be driven linearly 1020 along the axis and/or rotationally 1021 around the axis. In one embodiment, the cylinder may take the form of, or be coupled to, a leadscrew and/or leadscrew nut and used for 1-DOF translational motion along a screw axis and/or may be used to generate a differential drive enabling both linear and rotary motion along and about the axis, respectively. The cylinder may be a complete cylinder enabling 360 degree rotation or continuous rotation, or a partial sector for partial rotation.

In one embodiment, a plurality of such multi-directional elements (or sets of uni-directional elements arranged in at least 2 different orientation) are arranged around the cylinder, with their contact points in contact with the cylindrical friction surface and the cylinder can be driven translationally and rotationally. The cylinder may be a solid core, such as a drive shaft, or hollow core to enable an instrument or other object to pass through thereby comprising an actuator 1030

In one configuration, the actuator 1030 is used to rotate and/or translate a surgical instrument 1022 that passes through the hollow core 1014 that is driven by the elements. Such an actuator 1030 can be built into a robot, a modular removable robot end effector, or an instrument itself that attaches to a robot or other platform. In one example embodiment, this is used in surgical applications where an instrument, such as a directional ablation probe, can be inserted and rotated. The actuator 1030 may be integrated into an ablation probe (or other device such as a drill). In one embodiment, the actuator is incorporated into an active adapter is attached to the robot; this active adapter may be sterile and single use (and in one embodiment include an attached sterile drape or attachment for such) such that it couples to a robot on its proximal side and an instrument may pass through a hollow core actuated cylinder on its distal side.

In one embodiment, a cylindrical configuration comprises unidirectional (or multi-directional) piezoelectric elements that directly drive components of the robot. For example, a rotary joint may have a full or partial segment of a cylindrical surface and the tips of one or more piezoelectric drive elements may directly contact the surface of the robotic element, thus making it the friction surface. Note that the friction surface may be the curved surface, or a flat surface normal to it. In one embodiment, the contact point of tips of piezoelectric stacks such as, but not limited to, those from Piezomotor, DTI, Nanomotion, PI, or other manufacturers directly contact the surface of a rotating (or translating) element of the robot. Such a configuration enables much more compact mechanisms without the need for attaching separate independent motors. The contact surface of the robot may be modified to adjust stiffness or frictional properties.

Referring to FIG. 1I A further embodiment (comprising the alternate approaches described above) is a spherical version 1040 wherein the piezo element 1016 is on the surface of a complete or partial spherical friction surface 1041. The motor may rotate a sphere in up to 3-DOF, generating a full spherical wrist-like motion. In one embodiment, three or more such piezo elements 1016 surround the sphere and counterbalance the forces of each other, with appropriate preload onto the spherical surface.

### Example 2: Out-Runner Motor

We describe a mechanism capable of motion in one or more directions which may include rotation and translation along one or more axes. One embodiment of said mechanism includes an inner stator (201) which may include a toothed, smooth, or other shaped outer surface 210. Said stator 201 includes a piezoelectric (or other material of controllable strain such as a dielectric elastomer, electroactive polymer, photostrictive material, or other) element (203). Said element 203 can be excited as to induce vibrations or other mechanical deformations on the stator 201. Said vibrations or other deformations may couple, or otherwise contact or transfer energy, to a rotor (204) leading to motion of both or either stator or rotor. The inner portion (205) of the stator (201) may contain a through hole 306 through which other unrelated components can pass through or the inner portion 205 can be a solid component. This latter embodiment comprises all the previously described application including, but not limited to, passing a surgical instrument though the hollow cylindrical opening.

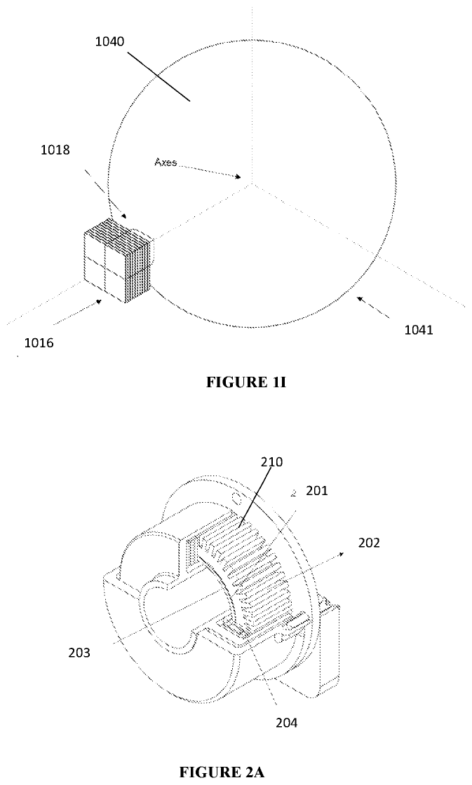

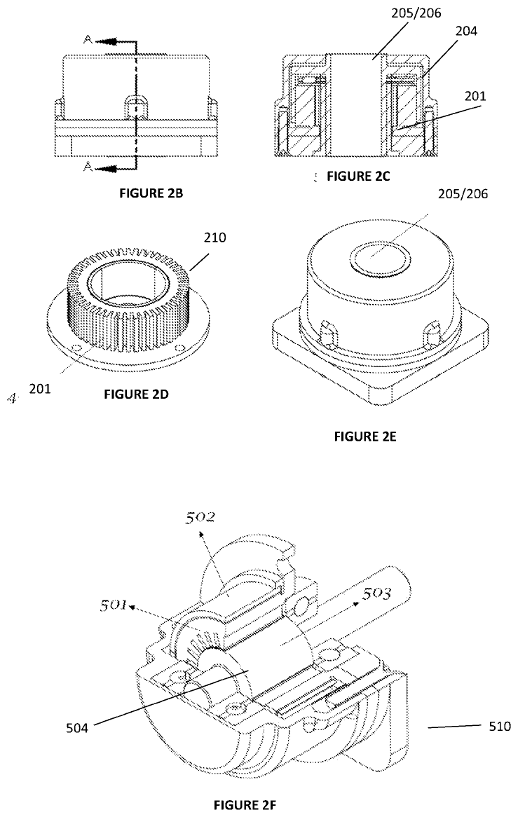

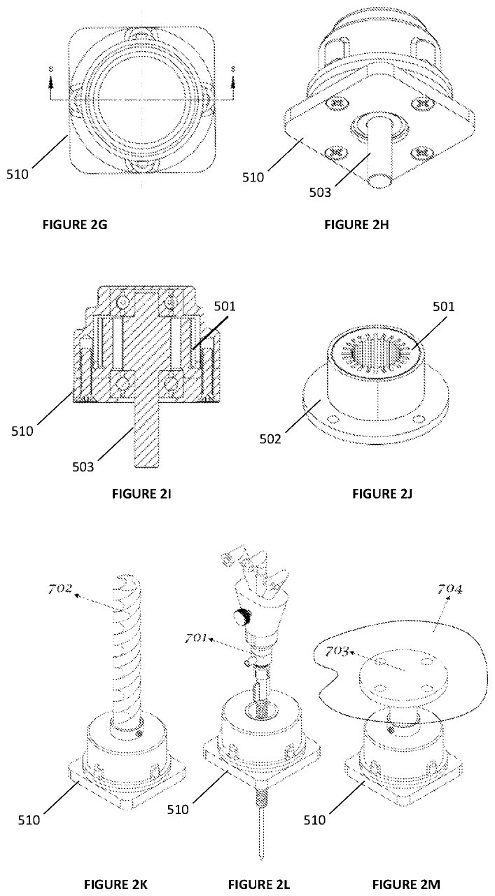

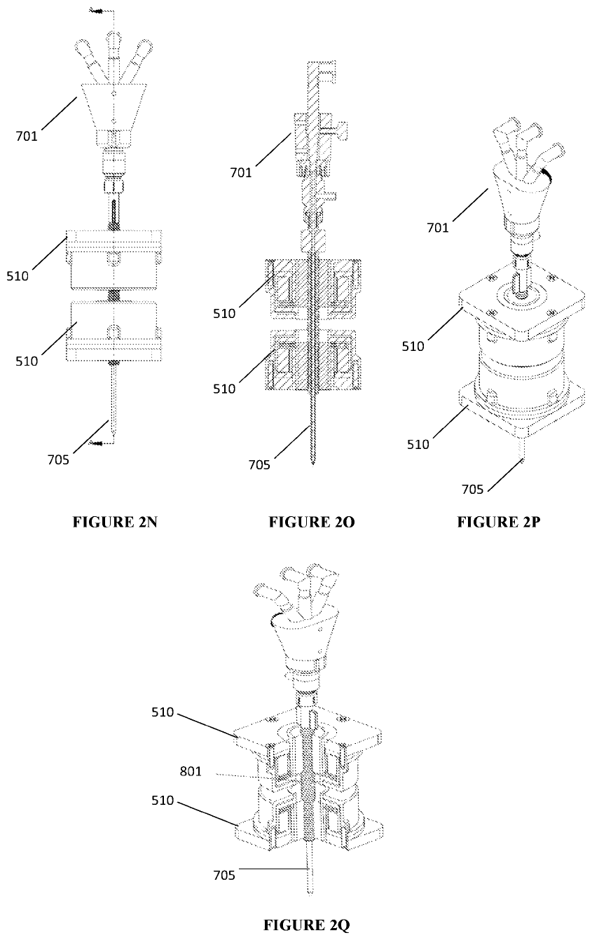

Referring to FIGS. 2A-E which show an exemplary configuration of one embodiment of an external hollow piezoelectric motor design.

In one example of example external hollow piezoelectric motor design, the rotor is copper or Ultem 1000, attached with PZT-5H ceramic. One of skill in the art will appreciate that any suitable conductors and piezo materials may be used.

This invention does not exclude the possibility where stator is excited in such a way as to generate rotational and translational motion simultaneously or in any combination thereof. This invention includes the possibility where the rotor is a part, a whole or otherwise connected to another unrelated device such as a probe for brain tumor ablation (701), drill (702), hub for attaching other devices (703) which may include a sterile drape (704), or others. Said components may be manufactured together or otherwise as a single or shared components through processes such as additive manufacturing, injection molding or others.

### In-Runner Motor:

In FIGS. 2F-M we describe a mechanism capable of motion in one or more directions which may include rotation and translation along one or more axis. This builds on the teachings above. Referring to FIGS. 2F-J one embodiment of said mechanism includes an outer stator (501) which may include a toothed, smooth, or other shaped inner surface. Said stator 501 includes a piezoelectric (or other material of controllable strain such as a dielectric elastomer, electroactive polymer, photostrictive material, or other) element (502). Said element can be excited as to induce vibrations or other mechanical deformations on the stator. Said vibrations or other deformations may couple, or otherwise contact or transfer energy, to a rotor (503) leading to motion of both or either stator or rotor. The inner portion of the stator (503) may contain a through hole through 504 which other unrelated components can pass through the inner portion 503 can be a solid component. This invention includes the possibility where stator is excited in such a way as to generate rotational and translational motion simultaneously or in any combination thereof. This invention can be used to provide linear and/or rotational motion to any device coupled to the stator or rotor. This invention includes the possibility where the rotor is a part, a whole or otherwise connected to another unrelated device. Specifically contemplated are devices such as a probe for brain tumor ablation (701), drill (702), hub for attaching other devices (703) which may comprise a sterile drape (704), or others. Said components may be manufactured together or otherwise as a single or shared components through processes such as additive manufacturing, injection molding or others. This invention does not exclude the possibility where more than one motor (510) is mechanically coupled or otherwise connected to a shared device or to each other. Said configuration may include mechanically coupling in such a way that differential actuation of the mechanisms lead to different directions or speed of motion.

FIG. 2M shows a hollow core cylindrical piezo motor 510, wherein the motor is built into the drape 704 (which can be sterile and single-use) and the probe/drill/tool can be put through the motorized hole and removably attached to it.

A drape adapter (i.e. active adapter) can be also attached to the motor. This approach may improve efficiency or have benefits in terms of sterility and convenience when used with surgical robots so as to enable rapid coupling of sterile actuated instruments.

FIGS. 2N-Q show an embodiment in which a plurality of piezo motors 510 a are coupled to an end effector 701 to drive the end effector. In the drawings an ablation probe is shown as the end effector.

Referring to FIG. 2Q which shows a differential drive system with two motors 510 driving an instrument (e.g. ablation probe). When the two motors rotate at the same speed and in the same direction, the instrument will rotate about the axis of screw 801. When the two motors rotate at the same speed but in opposite directions, the instrument will move linearly along the axis of screw 801. In other cases of varying speeds and direction the two motors will induce a combination or rotation and translation. Thus, this configuration enables control of the instrument to rotate and insert by controlling the speeds and direction of two motors. When using a differential drive arrangement such as this, typically one sleeve would have skew angled engagement, and one sleeve would have straight engagement (parallel to shaft axis) the skew angled engagement imparts a force tangent to the surface of the shaft, but in a screw pattern (along an angle). Holding the parallel shaft still, and rotating the skew engagement, the shaft will not rotate, because the parallel engagement is preventing it from rotating, so the skew engagement will drive it in a direction along the length of the shaft. When you drive both of them at the same speed, the longitudinal force is cancelled and the shaft moves in a purely rotary fashion. You can combine the relative velocities of these two engagements to provide an arbitrary combination of rotation and linear motion. These motors may be if a configuration previously described, or other configuration. The motors may be incorporated into the instrument itself, a modular end effector that couples to the instrument, a modular end effector incorporated into a sterile drape adapter, a robotic manipulator's distal end, or other configuration.

### Example 3: Robot Controller Configuration

One aspect of our invention comprises a portable robot controller 104 inside of the MRI scanner room. Note that in one embodiment this can be implemented with trunnion pins coupled to the rotating elements of motors 510 or equivalent structures riding in helical slots on the shaft (identified as screw 801). The robot controller 104 is RF/EMI shielded (which acts as a Faraday cage). The portable controller comprises internally low noise power supplies and is powered from a filtered AC input provided in the scanner room. It communicates to instrumentation outside the room (103, 108, 109, 110) utilizing a fiberoptic network connection that passes through a waveguide (105) between the MRI scanner room and an adjacent room (e.g. the MRI console area or equipment room). The instrumentation outside the room may include but is not limited to one or more of: networking equipment, a control computer, a navigation software interface, a planning software interface, a 3D visualization interface, the MRI scanner console, and a therapy delivery and monitoring system. This architecture is not tied to any particular room design or scanner vendor, and does not require modifications or specialized setup. The system may be used in more traditional diagnostic MR imaging suites or specialized interventional MR suites. The system can be rapidly brought into and setup in any MRI suite including interventional suites and diagnostic imaging suites. Embodiments of the system may also be used with other imaging modalities (e.g. ultrasound, fluoroscopy, and computed tomography) and can be used in a traditional Operating Room environment with at least some subset of the standard functionality.

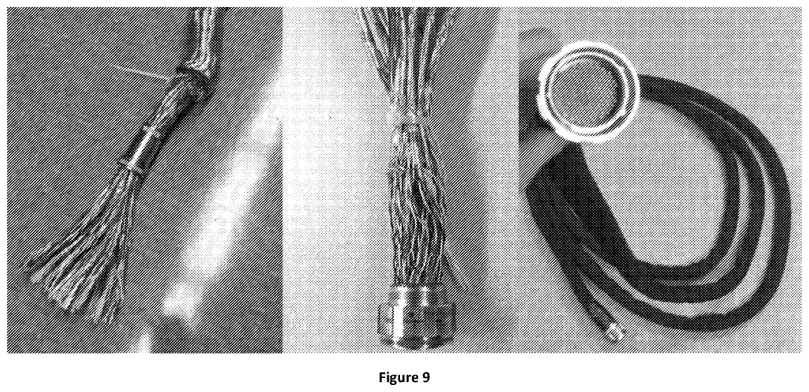

Referring to FIGS. 9A-C, in one embodiment, there is a single cable connection (107) between the robot controller (104) and the robot (106). The cable is a shielded cable that contains all power, communication, sensor, and motor drive signals. The cable may utilize electrically shielded non-ferrous avionics connectors on both ends. In one embodiment, the robot incorporates a breakout board and/or a pigtail to distribute the electrical connections on the robot. The single cable attachment significantly simplifies robot setup in scanner. This approach also enables modularity, wherein the robot may be configured with different options or modules. Or, further, the robot controller may be used with a variety of different robots.

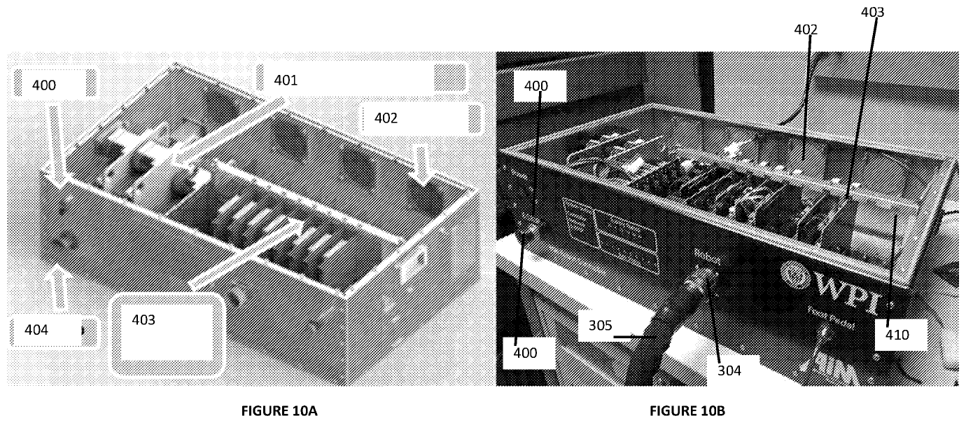

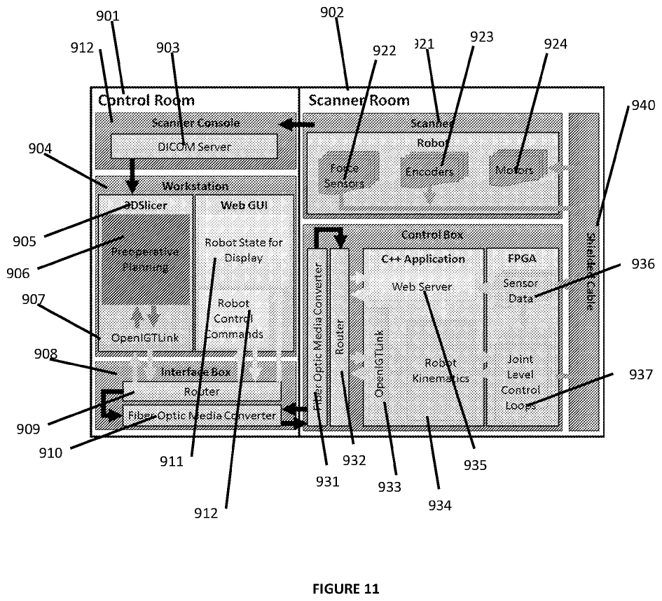

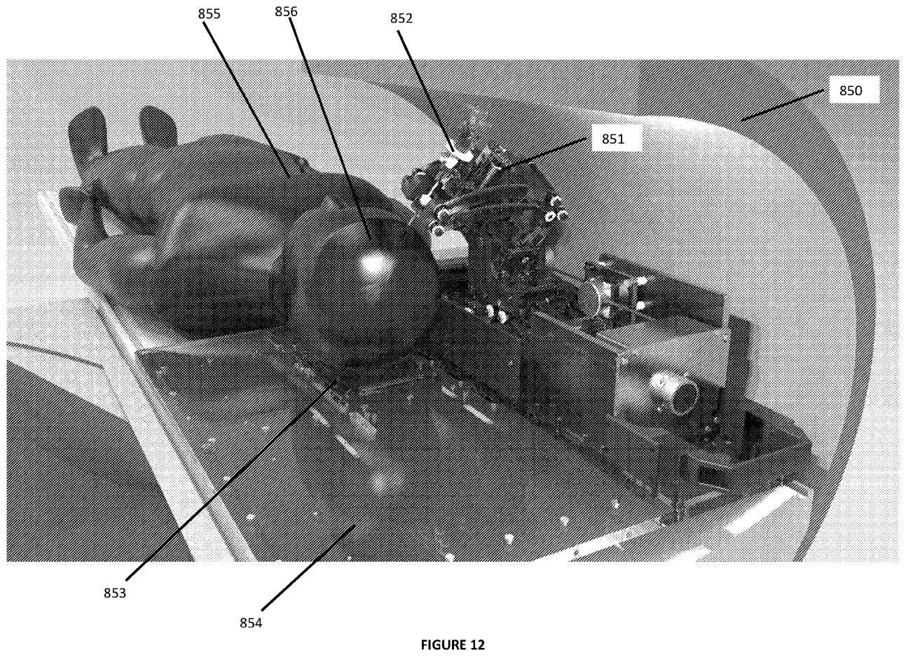

Referring to FIGS. 10A-B, an embodiment of the robot controller 399 has a modular configuration that enables the axes of the robot to be customized for a particular sensor and/or actuator configuration. Each axis has a corresponding card slot 410, and a card 403 that goes in slot 410 can be of various configurations. The cards can all include the same or similar front end including an FPGA or microcontroller for communication with the shared backplane over digital communication, including serial communication such as dedicated SPI busses per card slot. The cards can have different power output modules for driving various motors or other actuators, and/or can have various signal processing interfaces for reading encoders, potentiometers, limit switches, force sensors, fiberoptic sensors, or others.

Referring to FIG. 11 an embodiment of the robot controller supports up to 10 cards in 10 card slots. A robot-specific breakout board inside the robot controller takes the signals that go to/from the robot and route them to the connectors that goes to the common robot cable. An embodiment of the robot-side interface has a 8-channel breakout board for controlling 8 axes of motion. The cards that control motors can be utilized for a number of motors including piezoelectric motors. Cards can be designed to control harmonic and/or non-harmonic piezoelectric motors. Cards can support 2-channel, 4-channel, or other outputs. They can support high voltage or low voltage motor drive signals. They can provide sinusoidal, or arbitrary waveforms. They can be high frequency or low frequency. Example cards constructed and tested to date have been shown to support driving piezoelectric motors from Piezomotor, DTI, Shisei, Fokoku, Nanomotion, as well as custom piezoelectric actuators. These cards include encoder feedback for enabling closed loop control of position and/or velocity. An example force sensor interface card has been developed for collaborative control, and enables closed loop control of forces. This enables haptic feedback, puncture or interface sensing, sensory substitution, and admittance force control. This card can support one or more load cells with full Wheatstone bridges. Another embodiment of a force sensing card integrates fiberoptic fabry perot interferometry (FPI) sensing to measure force in one or more degrees of freedom (DOF). Alternatively, fiber bragg grating (FBG) or other optical sensing approaches may be incorporated. Control room 901 contains scanner console 912 containing an imaging server 903 connected to a workstation 904 having MRI (3D Slicer 905, and robot graphical interphase 913 having control 912 and status information 911. The workstation 904 is connected through a router 909 and fiber optic converter 910 to a scanner room 902 where the optical signal is converted back to an electrical signal through fiberoptic media converter 931 and fed to router 932 which is connected to control box 941 housing web server 935 and the robot kinematics 934. A shielded cable 940 passes signals to and from the robot sensors 922, encoders 923 and motors 924.

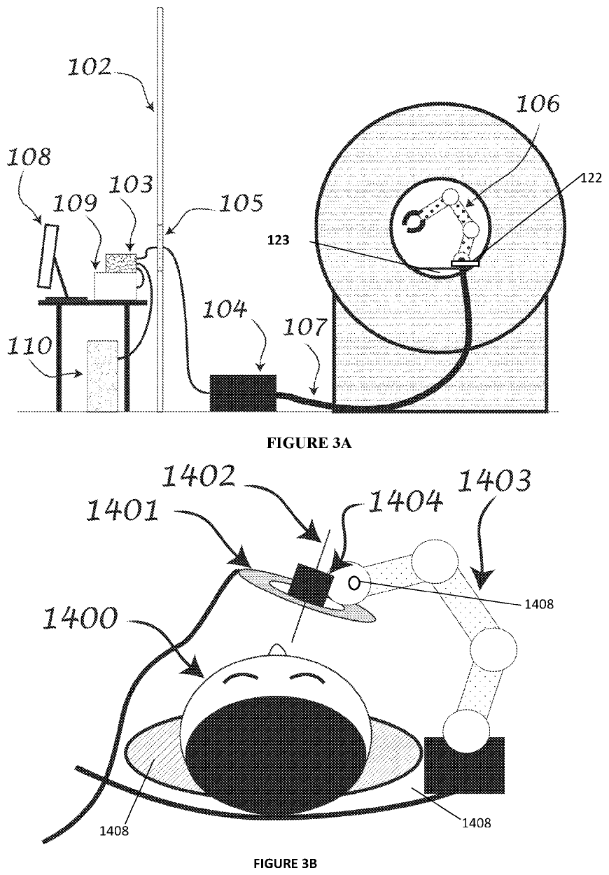

Referring to FIG. 3A which shows an embodiment of the robot 106 inside the bore of a diagnostic high-field MRI scanner 120. The robot 106 would reside inside the bore of the MRI 121 along with the patient and can be inside the bore and moving during active imaging. The robot 106 is connected to robot controller 104 via a cable 107. The robot controller 104 communicates with equipment in the MRI console area through a fiberoptic network connection that passes through the waveguide 105. The robot controller 104 can be portable and MRI-compatible and resides inside the scanner room; it requires no specialized MRI room configuration.

### Robot Configuration:

The robot 106 is designed to fit within the MRI scanner bore 121, and to do so with sufficient dexterity to maintain the necessary workspace while avoiding collisions. Optimizations have been identified to determine optimal parameters for a robotic mechanism given a desired workspace.

The robot can be configured to attach to a baseplate 122 that is removably mounted on the MRI scanner's bed 123. Baseplates may be scanner-dependent with a standard mounting interface for the robot to enable use on multiple scanner models and vendors. The patient's head 856 is supported on a head rest or skull clamp 853. The robot may be able to be reconfigurably attached to the base in multiple positions and orientations, such as being able to attach to both the left and right sides of the patient; this can improve reachable workspace. The robot may also be configured such that it can mount to a traditional operating room table. The robot and or its base plate may be configured to mount to other medical imaging or treatment systems.

The robot 106, the base plate 122, patient fixation 853 (e.g. skull clamp), and registration fiducial (e.g. a z-frame like fiducial or set of fiducial points) can be used as a single combined rigid unit.

### Integrated Head Imaging Coil:

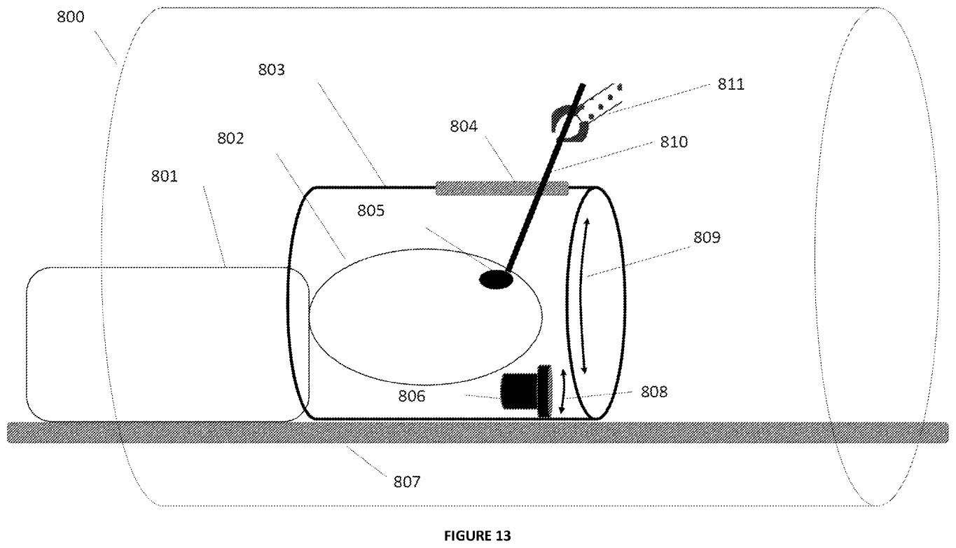

Referring to FIG. 13, in one embodiment a custom or modified imaging coil design 803 (e.g. head imaging coil) is integrated into the base platform and/or the robot. The robot is configured such that it can operate with a custom and/or modified head coil.

One embodiment of the invention includes an MRI imaging coil device comprising actuators. The imaging coils designed with one or more access port openings through the coil. The coil has the ability to change its position and or orientation with at least 1 degree of freedom of actuation. This motion of the coil is intended to align an access port opening through the coil with the intended trajectory or set of trajectories with which the robot can or will align the instrument.

Referring to FIG. 3B, the invention includes a system that combines imaging and manipulative abilities. Said imaging can be accomplished via incorporation of an MRI (or other medical equipment) coil (1401) (or other active or passive imaging component). Said manipulative abilities include mechanical, thermal or otherwise sharing or transferring of energy between the system and a patient or an object of interest (1400). Said transfer of energy may be accomplished via a robotic manipulator (1403) and or some end effector (1404). An embodiment of said invention may include an MR imaging coil that incorporates a single or multi degree of freedom actuator for positioning of another device within its field of view. Referring to FIG. 13, the imaging coil 803 is designed to at partially or fully enclose a part of a patient, in the figure it is a head 802. Access ports 804 are placed in the imaging coil to allow instruments 810 and/or a robot 811 to reach the patient during a procedure. A motor 803 is capable of rotating the coil 803 around the patient. Other degrees of motion can be provided.

### Registration and Tracking:

Referring to FIG. 3B, in one configuration a tracking fiducial 1408 is integrated into the base platform so as to define the position and orientation of the robot with respect to the MRI scanner and its imaging coordinate system. The fiducial may enable 6-DOF localization with one or more images, such as with a z-frame type fiducial, typically comprising 7 or 9 tubes that are imaged with cross-sectional MRI slices or 3D volumetric imaging. The fiducial localization software may have the ability to find a limited number of the DOF of the position and orientation when certain DOF are known. In one example, the base platform is attached to the top surface of the scanner bed in slots, and only the axial translation along the bed may need to be assessed. In this scenario the fiducial may only be a single element such as a vertical tube imaged with coronal slices. The fiducial may be integrated into the base platform, or it may be removably but repeatably mountable to the base platform so as to use it for registration and then remove during the procedure. The fiducial may be filled with a liquid or gel that has high imaging contrast, such as including gadolinium or other compounds.

An embedded fiducial in the robot 1407 for localization and/or confirmation. A fiducial on the robot may be used for initial registration of 6 DOF or a subset thereof. The embedded fiducial may take the form of a tube aligned with the axis of the instrument or cannula being held by the robot, wherein the fiducial may be imaged to show up as approximately a circle with a dark center when imaged with slices orthogonal to said axis.

One or more compact imaging coils may be used as active tracking coils. These coils may be integrated into the instrument (i.e. the tool being placed), the cannula, or a component of the robot's end effector. They can be used to generate high contrast images quickly. They may support projection images (i.e. very thick slices) for rapid localization of one or more points.

An approach for localizing (position and or orientation, or some subset thereof) the robot, an instrument, or other object in the MRI based on the map to a unique field inhomogeneity (or a pattern there of).

### Visualization, User Interface, and Surgical Navigation:

Integration of augmented reality, virtual reality, and/or mixed reality into surgical planning. Visualization of a simulation of the virtual robot following its intended trajectory in virtual representation of the medical image data. A live update as the robot moves so as to confirm a safe motion.

An MRI-compatible augmented reality, virtual reality, and/or mixed reality headset, glasses, or similar interface. The device configured to represent the proposed robot motion plan on the surgical workspace. The device configured to show the anatomy of the patient, based on medical image data, on the surgical workspace.

A handheld MRI-compatible pendant for control and/or status updates from the robotic system.

An LED or display on the robot that allows the surgeon to understand the robot's next move and/or status

### Integrated Closed Loop Control of Ablation:

An ablation probe can be precisely controlled using feedback from the MRI. In this method thermal imaging within the MRI is used to provide closed loop control of the ablation probe using temperature feedback based on MR thermal imaging to provide control of the lethal thermal boundary. Using modeling and model-based control to ensure precise thermal dose delivery. This can be in addition to or in replacement of temperature sensor in the ablation probe.

In a preferred embodiment synchronization of robot movement (and possibly encoder) to MRI machine occurs such that the robot does not emit or record during the acquisition or RF pulses.

### MRI Induced Heating or Shape Changing:

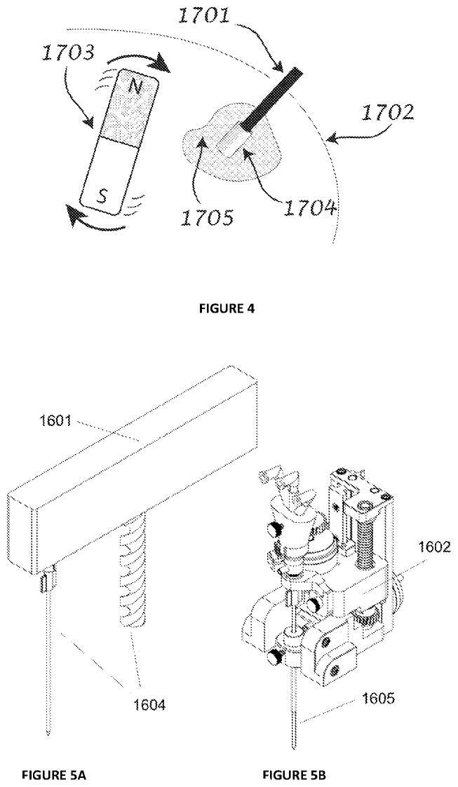

Referring to FIG. 4, we describe a device for localized heating (1705) of an object of interest (1702) inside a magnetic field. One embodiment of said device may constitute a ferromagnetic or other conductive component (1704) capable of interacting with magnetic or electromagnetic fields. This invention does not exclude the possibility of generating localized heating via quick oscillations or vibrations induced by changing gradients (1703). This invention does not exclude the possibility of using an MRI for tracking of said device within a body and use of specific MRI scanning sequences for inducing of said heating. Said heating, may also be used for activation or deployment of said device such as the case in which the device is designed to change shape via usage of a shape memory alloy or other thermal-shape relationship. Said heating, may also be used to deteriorate or otherwise destroy the device for localized drug delivery or safe disposal. This invention includes the possibility where the device is attached to a delivery mechanism (1701). This delivery mechanism may be a robotic device operating within the bore of an MRI machine, a catheter, a guide wire, or a needle.

An example of an embodiment of utilizing the switching gradients of the MRI to induce vibration of an element that in turn causes localized heating. Such heating may be precisely modeled so as to form specific shaped lesions. It may also be monitored utilizing MR thermal imaging (MRTI) to monitor, and in some cases provide for closed loop control of, dose delivery.

### Example 4: Modular End Effectors, Single Patient Actuated End Effectors

Modular end effectors that couple to a base portion of the robot. The modular end effectors may be active or passive. In one scenario the modular end effector is a simple guide sleeve and/or cannula that couples to the robot, such as for guiding a particular tool. I another scenario the modular end effector has integrated one or more DOF of actuation. The modules may include one or more of instrument insertion, cannula insertion, and instrument rotation. One or more of those DOF may be integrated into the modular end effector. Some DOF may be part of the robot and others as part of the end effector.

A method for connecting the modular end effectors. The end effectors are single patient use and provided in a sterile kit. The robot is draped with a sterile drape, and the end effectors are attached to the robot from the outside of the sterile boundary. The sterile drape may be a specialized drape with a mounting plate for connecting to the robot on one side and the end effector or component thereof on the other side. The mounting plate may contain electrical connections for passing electrical signals from the robot to the active end effector which is in the sterile field.

An active end effector module with at least 1 DOF of actuated motion. The motion provided by piezoelectric actuators. The piezoelectric actuator comprises a piezoelectric ring that enables an instrument to pass through the center. The module may further comprise sensing such as optical encoding of absolute or relative position. The end effector module configured to be a single patient use (i.e. disposable) device that enables rotation of the instrument.

An injection moldable plastic actuator (i.e. motor) incorporated into an active end effector module (e.g. a directional ablation probe or other therapy delivery device. Such a design of incorporating the actuator into the instrument or an instrument holder that is part of the sterile kit reduces the DOF of the robot itself and improves the workflow through ease of maintaining a sterile field even with an active end effector.

A drill, cannula, ablation, biopsy, or other instrument that is a readily removable/attachable (i.e. hot swappable) end effector allowing attachment through the sterile barrier. The end effector may contain electronics, and said attachment through the sterile barrier may comprise one or more electrical connections. The end effectors may be designed to be for single use, single patient use, limited lifetime, or durable.

A sterile kit comprising an ablation probe that integrates a piezoelectric actuator. Said piezoelectric actuator provides for controlled rotational motion of the probe about its axis. The piezoelectric actuator being of a hollow or open center so as to be concentric with the probe.

### Swappable End Effector:

Referring to FIGS. 5A and 5B, we describe an apparatus capable of holding different modular tools inside strong magnetic fields of devices such as a magnetic resonance machine. Said apparatus can swap the end effector, or other tool, with or without intervention of another device or person. Said apparatus consists of a mechanism (1602) for holding the tool (1605) while in use and another mechanism (1601) for holding one or more tools (1604) that are not in use. The invention does not exclude the possibility where the apparatus can stow a tool back into the mechanism for holding one or more tools after use. This invention includes the possibility where the tools can only be passed from the tool holder to the in-use tool holder and used tools are discarded. The invention does not exclude the possibility where the apparatus consists of a single mechanism that holds a multitude of tools simultaneously with the necessary motions for using the tools as required by the application.

(Left) Swappable tool mechanism holding one or more tools or end effectors. (Right) A modular tool end effector attached to a distal end of a robotic manipulator.

### Example 5: Single Coax Probe

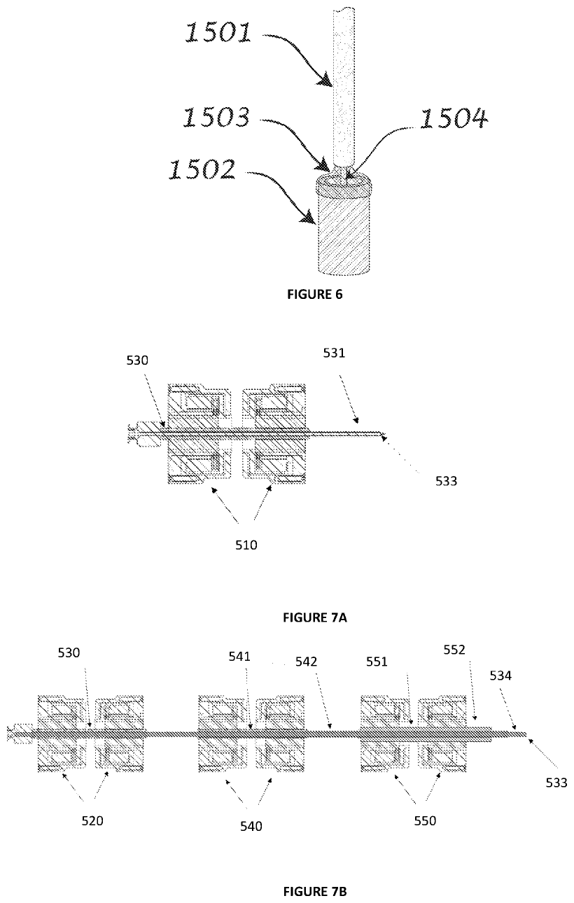

In FIG. 6, we describe an instrument, including but not limited to an ablation probe, consisting of a piezoelectric element (1501) supported by a rigid or semi-flexible body where energetic (electric or optical) coupling between a controller and said piezoelectric element is made via a single coaxial cable (1502, 1504). This invention includes the possibility of using the outer shielding of the coaxial cable (1502) in such a way as to have a complete shielding (1503) from cable origin until end of piezoelectric element (1501).

### Example 6: Steering

A modular end effector comprising actuators providing a translational motion and a rotational motion about the axis of the translational motion. Said end effector configured to steer an asymmetric tipped instrument (i.e. bevel tipped needle) though tissue by adjusting rotation and insertion in a coordinated manner. The actuators may be piezoelectric actuators, and may be configured as a ring concentric with the rotational motion wherein an instrument passes through the ring. A configuration in which two such piezoelectric actuators are utilized in a differential drive configuration wherein a coupled motion provide rotation and a coordinated differential motion provides translation and/or rotation. Examples of steering are taught in U.S. Pat. No. 10,052,458.

Referring to FIG. 7A, an example with an asymmetric tipped instrument such as, but not limited to, a bevel-tipped needle. The needle 533 inserted into a drive shaft 530, wherein the drive shaft 530 may be part of the needle or part of a needle driver module that the needle is inserted into. The drive shaft 530 is threaded 535 and passes through two hollow core motors 510. Alternatively, the motors shown could be cylinders, gears or other objects driven by adjacent motors through gears, belts, etc.

In the differential drive system with the two motors 510 driving the instrument (e.g. needle or ablation probe), when the two motors 510 rotate at the same speed and in the same direction, the instrument will rotate about the axis of drive shaft. When the two motors rotate at the same speed but in opposite directions, the instrument will move linearly along the axis of drive shaft. In other cases of varying speeds and direction the two motors will induce a combination or rotation and translation. Thus, this configuration enables control of the instrument to rotate and insert by controlling the speeds and direction of two motors. This motion can be used in steering the needle path inside of the tissue.

Another embodiment comprises a modular end effector comprising a plurality of concentric rotations and translational motions along the axes of rotation. Said end effector configured to manipulate nested concentric tubes. Said tubes may be pre-trained curved nitinol tubes and/or wires that can follow a particular path through tissue by moving the translations and rotations in a coordinated manner.

FIG. 7B, shows an embodiment comprising a number of concentric tubes/needles. Each tube or needle may be pre-formed or trained to a particular shape which may be a constant curvature arc for part of its length. Each tube or needle may be moved one or two DOF (insertion, rotation, or both). In one embodiment, as shown, three separate elements each have two-DOF insertion and rotation capabilities, and each has a pre-trained curvature, thus allowing steerability of the needle tip. In this embodiment a first set of hollow core motors 510 are connected to a drive shaft 530 which drives an inner most tube or needle 534, a second set of hollow core motors 540 drive a drive shaft 541 which is coupled to intermediate tube 542; a third set of hollow core motors 550 connect to drive shaft 551 which is coupled to an outermost tube 552. It will be appreciated that the number of motors and tubes can be increased or decreased as needed for a specific application.

The approaches outlined herein lend themselves to disposable instruments which avoid the cost of cleaning and the uncertainly of proper sterilization between patients. A single-patient use steerable instrument end effector module comprising multiple piezoelectric actuator rings can readily be produced. In one embodiment, the hollow core motors are very low cost rings and can be integrated into single use instruments including, but not limited to, ablation probes (e.g. ultrasound-based, laser-based, cryo-based), neuromodulation probes, injection or other therapeutic delivery instruments, dexterous instruments, co-axial needles, etc. Such instruments can play an effective role in preventing infection in a medical setting.

### Embodiment 6: Active Sterile Drape

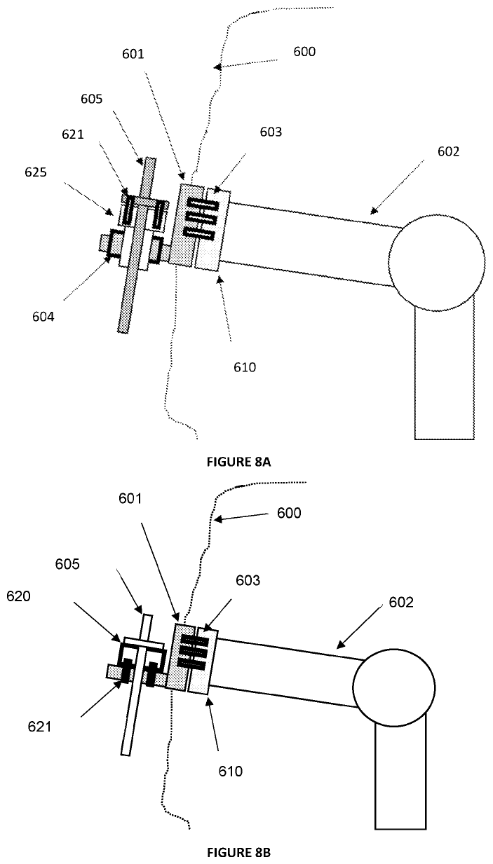

Referring to FIG. 8, this embodiment comprises a sterile drape 600 comprising one or more actuators. In a most preferred embodiment the drape and actuator(s) are intended to be for single patient use. The sterile drape is designed so as to cover the durable and/or nonsterile portions of the robot. A mounting plate 601 is integrated into the drape 600 that couples to the robot/manipulator 602. Said coupling including both mechanical attachment and electrical connections 603. The coupling may also include pneumatic, hydraulic, fiberoptic, or other connections. The coupling may also include direct mechanical coupling such as a pushrod or driveshaft.

The exterior sterile surface of the active sterile drape comprises one or more motorized actuators 604. In one embodiment, the exterior surface of the drape comprises a hollow core rotary piezoelectric motor 604. Said motor has one of its components (e.g. stator side) fixed to the drape's mounting plate 601, and another surface (e.g. the rotor side) further comprising a means for mounting a surgical tool, probe, cannula, or other instrument 605. The unit may further incorporate encoding via optical encoders or other means for determining the absolute and/or relative position of the motor.

The unit may further comprise integrated force and/or torque sensing. In one embodiment, the unit comprises a 6-DOF Force-torque sensor. In other embodiments, a subset of those DOF may be implemented. In one configuration, said force and/or torque data is used for hands on cooperative control of the robot, wherein the robot motion is controlled by interaction forces and moment applied to it. The force and/or torque sensor data may also be used for determining contact with an object, such as for safety; it may be used for assessing or identifying tissue boundaries: it may be used for characterizing tissue (e.g. identifying tissue stiffness as a probe is inserted. The sensor may be electronics with electrical signals passing through the sterile drape's adapter plate. The electrical signals may be analog signals, or onboard signal processing may occur on the distal module and digital signals are passed through the adapter plate (e.g. serial communication). In one embodiment one or more DOF of force and/or torque sensing is based on fiberoptic means (e.g. FPI of FBG) and the optical signal passes through the adapter plate.

The adapter plate of a sterile drape may further comprise auxiliary connection 603. In one embodiment the adapter plate enables passing electrical signals for powering piezoelectric elements of an ultrasonic interstitial thermal ablation probe as well as associated liquid cooling supply and return lines.

In another embodiment, the sterile drape comprises a mounting plate that passes the connections identified above through, but rather than containing one or more actuators it comprises a mounting plate 601 upon with a sterile motor or other actuator can be mounted. The mounting plate may also enable motor-integrated instruments such as an ablation probe containing a motor to be mechanically and electrically coupled to the robot.

An example configuration showing a manipulator that may be a robotic manipulator, a passive arm, or other instrument holder. In one configuration it is an MRI-compatible robot that operates inside the bore or an MRI scanner. In another embodiment it is a teleoperated surgical robot. The manipulator has an interface at its distal end for coupling to an active sterile adapter 601. The active sterile adapter 601 comprises one or more active components including, but not limited to, motors 604. The active sterile adapter comprises a sterile drape 600, or it comprises a means for coupling to sterile drape. The means for coupling would entail an opening in the drape which would be attached to a mounting plate via adhesives, tape, mechanical fasteners such as threaded fasteners and/or rivets with or without a backing plate; any means of fastening which is acceptable to a regulatory authority may be used, The adapter may have one or more connections 603 to the robot/manipulator 602; these connections may be mechanical, electrical, optical, pneumatic, hydraulic, and/or other types. In one embodiment, the active sterile adapter mechanically couples to the manipulator and the connections include electrical signal to drive a motor integrated into the active sterile adapter. The active sterile adapter may comprise one or more motors. In one embodiment this motor is a hollow core motor. In a further embodiment the motor is a piezoelectric hollow core. In one embodiment, the integrated motors are two coaxial motors configured in a differential drive configuration to enable insertion and/or rotation of an instrument. In another embodiment, the integrated motors are a plurality of motors for controlling dexterous manipulation of an instrument. These motors may be in runner, out runner and differential drive motors as disclosed in this application. An instrument is coupled to the active sterile adapter. In one embodiment, the system is modular and can accept a variety of different instruments. In another embodiment an instrument is permanently coupled to the active sterile adapter and they act as a single unit. The instrument may have mechanical, electrical, optical, pneumatic, hydraulic, and/or other types of couplings to the active sterile adapter though the instrument coupler. In an alternate embodiment, a cable connects the instrument to the active sterile adapter, the manipulator, a control system or other external device directly.

In one embodiment of the system, the instrument is an interstitial therapeutic ultrasound probe. The probe couples to the motors and passes through the hollow core that enable rotation and or insertion. Motor power and encoder feedback signals pass through the adapter connections and are powered by the signals running to the manipulator, which takes the form of a robotic manipulator. The instrument receives electrical signals as well as cooling liquid flow directly from an external source.

One of skill in the art will readily appreciate the teaching herein can be applied to develop multiple devices embodying the disclosed inventions.

We claim:

1. An omnidirectional piezo motor comprising:
a) at least two piezo electric stacks, each stack comprised of a plurality of piezo electric crystals each crystal having a conductive and a non conductive region, where in the stacks are oriented so the non-conductive regions of each stack are adjacent to each other;
b) a controller electrically connected to each of the at least 3 stacks for providing a controlling electrical signal to each piezo electric stack

2. The omnidirectional piezo motor of claim 1 where in the stacks are controlled via waveforms.

3. The ominidirection piezo motor of claim 3 wherein the waveforms are sine or cosine.

4. The omnidirectional piezo motor of claim 3 wherein multiple piezo electric stacks are combined and controlled to allow piezo electric static to enact a desired motion in more than a single axis.

5. The omnidirectional piezo motor of claim 4, where the piezo electric motor is comprised of 4 pillars.

6. The omnidirectional piezo motor of claim 5 wherein the pillars are capped with purpose specific tip.

7. The omnidirectional piezo motor of claim 6 where the pillars can be coupled and actuated in unique ways to enact driving force in an arbitrary direction within a plane tangent to a point on the actuator tip.

8. The omnidirectional piezo motor 7 where the pillars can be coupled and energized in unique ways to enact a driving force within any a contact surface in contact with the actuator tip and the force is acting in a direction tangent to that point of contact and perpendicular to the normal axis of contact

9. The omnidirectional piezo motor of claim 8 wherein the contact surface being acted upon is a known geometric shape, including but not limited to circles, spheres, cylinders, rectangular prisms and others.

10. The omnidirectional piezo motor of claim 1 wherein the piezoelectric crystals are arranged radially as a stator and act upon a rotor.

11. The omnidirectional piezo motor of claim 10 wherein the center of the motor is hollow.

12. The omnidirectional piezo motor of claim 10 where multiple radial actuators can be arranged and controlled in concert to generate complex motions.

13. The omnidirectional piezo motor of claim 12, where multiple motors are arranged on a single axis to function in a differential motion system.

14. The omnidirectional piezo motor of claim 11, comprising:
a) a first piezo motor acting on a first drive shaft which is coupled to a first control element,
b) a second piezo motor acting on a second drive shaft which is coupled to a tube concentric with the first control element;
c) wherein the tube and the control element are capable of individually controlled motion.

15. The omnidirection motor of claim 14 further comprising one or more additional motors each acting on an additional driven tube, all of the tubes being concentric with the first control element and the concentric tube.

16. A method of operating the omnidirection motor of claim 1 wherein the different piezo crystals, stacks, and arrangements are energized in concert in a coordinated fashion such to facilitate a coordinated, complex motion.

17. The omnidirectional motor of claim 11 wherein the hollow center of the motor is designed to hold a tool or instrument.

18. A system of tools and or improvements to enable MRI guided interventions, comprising a modular and configurable architecture; said architecture separating functional elements for MRI guided technologies. This includes:
a. Modular controller configuration, such that swappable card based drivers can be interchanged to operate a variety of MRI compatible actuators, sensors, and interface devices.
b. An MRI compatible central computing/controller system
c. An MRI compatible communication route through the patch panel of the control room to connect to a non-mri compatible user interface;
i. User interface could be a standalone computer;
ii. User interface could be directly tied to control computer.

19. A module for the system of claim 18, wherein the module acts as an MRI compatible motor driver, specifically paying attention to driving signal and communication signal generation such as not to emit intolerable interference into the scanner

20. The module of claim 19 wherein the module is for controlling a piezo electric ceramic motor or arrangement of piezoelectric motors

21. A module according to claim 18, wherein the module can operate and interface with an imaging coil within the scanner or a robot within the scanner.

22. The module of claim 21 wherein the imaging coil is mounted on the robot to move with the robots range of motion specifically to effect the image optimally based on the motion of the robot.

23. A module according to claim 18 in which the module registers the position of equipment within the imaging space.

24. A module according to claim 23, in which the module is a contrast agent filled fiducial such that it can be segmented in the imaging space.

25. A module according to claim 24, which is permanently mounted with a known geometric relationship to the robots coordinate system such that the robots coordinate system can be registered within an imaging volume.

26. The registration fiducial of claim 24, where the robot system or base system can be attached to at least one other structure in the scanner room such as the bed rail, such that the fiducial can be used to track the relative position of said structure to the imaging space.

27. A module of claim 18, wherein the module is a user interface or display wherein the module could be an alternative to a computer screen.

28. The module of claim 27, wherein the module is a virtual reality headset, that allows the user to view representative imaging in 3 dimensional space, and utilize head tracking to optimize availability of data and minimize required use of hands.

29. The module of claim 18 wherein the module is an augmented reality headset such that the user can view additional information from the system, as well as transparently see the environment around or in front of them.

30. A module of claim 18 wherein the module is a sensing receiver to provide the control system information from various devices.

31. The module of claim 30, wherein the sensor is providing information on thermal properties of an ongoing procedure.

32. The module of claim 31 wherein the sensor is a thermistor, thermal probe, or MRI based temperature readings.

33. A method for operating the system of claim 18 in which the system and modules are controlled to perform complex operations by utilizing some or all of the subsystem components and modules in a coordinated fashion.

34. A method according to claim 33 wherein thermal information is utilized to perform closed loop thermal control of a thermal tool such as an ablation probe.

35. A module of claim 18, where the module's physical properties can be influenced by a scanner magnetic field and alteration thereof.

36. A module as described in 35, in which the module responds to motion or vibration induced upon it by integrated control of the scanner system magnetic field such as controlling frequency, intensity or direction of magnetic field.

37. The module of claim 34 in which the module generates heat in response to the MRI field modulation.

38. A module for the system of claim 18 comprising a modular end effector to the robot, also known as end of arm tooling.

39. The module of claim 38 wherein the module is a swappable end effector capable of being replaced with a minimum of tools

40. The module of claim 39 where the module is swappable during a procedure to expand the capabilities of the robotic system.

41. The module of claim 39 wherein the module is a biopsy needle, bone drill, or other device that either is, or replicates the function of standard surgical tools.

42. The module of claim 18, wherein the module is a coaxial shielding extensible across the coupling of multiple physical modules to maintain an RF shielding barrier.

43. The module of claim 42 wherein the module is a conductive flexible sleeve and a mating agent.

44. A module of claim 43, wherein the module contains components or structure beneficial to be MRI shield, which can interface and extend the MRI shield through application of the interface.

45. A module of claim 41, wherein the module is a needle driver.

46. The module of claim 45 wherein the needle driver comprises hollow core piezo electric motors with the shaft advancement mechanism contained within the hollow core drive.

47. A module for the system of claim 18, wherein the module is a sterile drape incorporating one or more active or inactive elements and features to allow functionality to penetrate the sterile barrier, wherein the drape comprises an interface and connection point, having at least one conductor, actuator, sensor or pass through.

48. The module of claim 47, wherein the module is disposable or single use, and designed to be discarded or reprocessed after each patient.

49. The module of claim 47 wherein module is customized for specific patients or procedures.

50. A module such as in 47, wherein the interface for the active surgical drape can be used as a common interface for swappable end effectors.
